(19) United States
(12) Patent Application Publication
(10) Pub. No.: US 2007/0299525 A1
(43) Pub. Date: Dec. 27, 2007

(54) Bone Spacer

(75) Inventor: Radames Binotto, Battaglia Terme (IT)

Correspondence Address: Themis Intellectual Property Counsel, 7660 Fay Ave Ste H378, La Jolla, CA, 92037, US

(73) Assignee: BIOMEDICA S.R.L., Battaglia (PD) (IT)

(21) Appl. No.: 11/573,040

(22) PCT Filed: Jul. 25, 2005
(86) PCT No.: PCT/IT05/00439

§ 371 Date: Feb. 21, 2007

(30) Foreign Application Priority Data
Aug. 5, 2004 (IT) PD2004A000213
Jul. 19, 2005 (IT) PD2005A000225

(51) Int. Cl. A61F 2/44; A61F 2/28

(52) U.S. Cl. 623/17.16

(57) ABSTRACT

This is a new bone spacer with semispherical projecting parts and/or teeth suitable for fitting and engaging in cavities specifically prepared on the surface of the vertebrae so as to prevent the displacement of said spacer, and containing at least one channel placing the side facing towards the operator during its insertion in communication with the two opposite, superior and inferior surfaces facing the vertebrae. Plates are also provided for coupling superiorly and inferiorly to the spacer, complete with ribs and/or teeth.

This patent relates to the field of medical devices for spinal neurosurgery, neurotraumatology, orthopedics, and particularly concerns bone spacers, i.e. medical devices used for intersomatic spinal fusion with a view to restoring sagittal balance and generally for conditioning the spinal column.

Surgical operations are generally associated with a loss of substance, which needs to be reintegrated with biocompatible material.

In the case of vertebral pathologies, in particular, it becomes necessary to use bone spacers in order to obtain cervical fusion at one or more levels for the treatment of degenerative and traumatic pathologies.

Organic and synthetic bone spacers are currently used.

A first problem common to the spacers in current use lies in that the spacer occludes the Haversian canals that enable the nourishment of the bone tissue, thus giving rise to an undesirable high intraoperative morbidity.

Moreover, the spacers generally have a low mechanical strength and succeed in assuring only a limited degree of intersomatic fusion.

Some spacers of known type are complete with holes, which reduce the risk of occlusion of the Haversian vessels and guarantee a greater degree of fusion.

All the known spacers nonetheless share the drawback of being impossible to position stably from the start, thus carrying the risk of the spacer's final position being suboptimal.

To overcome all the above-mentioned drawbacks, a new type of bone spacer has been studied and realized, that is particularly suitable for the purposes of cervical decompression.

The object of the new spacer is to enable its stable initial positioning, i.e. to ensure primary immobilization.

Another object is to enable a high degree of intersomatic spinal fusion, i.e. to guarantee secondary immobilization.

Another object is to offer a sufficient degree of elasticity between the new spacer and the vertebrae between which it is inserted.

Another object is to limit the occlusion of the Haversian canals.

A further object is to offer a mechanical strength comparable with that of the vertebrae.

These and other, direct and complementary objects are achieved by the new bone spacer complete with holes for the passage of blood and for enabling the growth of bone trabeculae inside said holes, and with projecting parts suitable for fitting and engaging in cavities specifically created for said purpose on the surface of the vertebrae, and with further projections, or teeth, suitable for preventing the displacement of said spacer, and having at least one channel communicating both with the side facing towards the operator during the placement of the spacer and with the two, superior and inferior surfaces facing the vertebrae between which said new spacer is inserted.

The new spacer is made of a biocompatible material and consists of a device that has a preferably parallelepiped shape and is complete, on each of the two surfaces facing the vertebrae, with means for enabling the primary and secondary immobilization of said spacer, and that has one or more channels that are accessible to the operator during the insertion of the new spacer between the vertebrae and that communicate with at least one of the surfaces facing the vertebrae between which said new spacer is placed.

Each spacer surface in contact with the vertebrae has radial grooves or cavities communicating with the exit from said channels.

To be more precise, each surface has at least one semispherical projecting part suitable for an easy and stable engagement of the spacer.

Moreover, the surfaces of said new spacer also have further means for preventing the displacement of said spacer, in the form of projections, or teeth.

Each of said projections can be positioned so that they coincide on either side of the device, i.e. the spacer is symmetrical, or their positions may not coincide points, i.e. they occupy different places on either side, so the spacer is non-symmetrical.

The new spacer also includes one or more sets of holes to enable blood flow and the formation of bone trabeculae inside said holes, thus assuring the secondary immobilization of the spacer, i.e. the fusion of said spacer with the adjacent bodies.

Said holes are preferably located as uniformly as possible.

Provision may be made for said new spacer not to have said projecting parts, and being coupled instead, on one or both surfaces facing the vertebrae, by means of corresponding metal plates suitable for enabling and accelerating bone fusion. For this purpose, the new spacer is made with parts in relief or ribbing on the surfaces facing the vertebrae, suitable for coupling with corresponding grooves provided in said plates.

Said plates have corresponding projecting parts for primary and secondary immobilization, and may also have one or more holes communicating with the channels in the spacer.

The new procedure for fitting the new spacer involves the surgeon milling the adjacent vertebrae before inserting said spacer, so as to create a cavity in said vertebrae suitable for containing said semispherical projecting part. Thus, once it has been positioned with said semispherical parts engaged in said cavities, the spacer remains stable.

That is to say that primary immobilization is guaranteed.

If a spacer with channels is used, once the new spacer has been inserted in position between the vertebrae, the operator injects some adhesive material and/or diversified stem cells, or other types of gel or fluid material into the aforementioned channels.

Said injected material reaches and fills the gaps between the new spacer and the vertebrae in between which said spacer is placed, thereby ensuring the bond between the new spacer and the vertebrae.

The material used to manufacture the new spacer must have mechanical features similar to those of the adjacent vertebrae, and must become perfectly integrated with human tissue.

Examples of materials suitable for said purpose include: hydroxyapatite mixed with plasma, bioceramic glass suitable for bone fusion, metal products of the type used in implants, e.g. titanium, medical steels, etc, medical elastic, plastic or silicone materials, and also ceramic materials, synthetic and natural polymers, resin tissue, or polyethylene, suitable for injecting cells capable of colonizing and converting the support material.

Bone spacer suitable for inserting between two adjacent vertebrae, consisting of two surfaces suitable for coming into contact with said two vertebrae and complete with holes, with a preferably semispherical protuberance on at least one of said surfaces suitable for engaging in a cavity specifically prepared by milling on at least one of said vertebrae, and also with a channel suitable for placing the surfaces of said new spacer facing towards the vertebrae in communication with the side of said new spacer that remains accessible to the operator during the insertion of said new spacer between said vertebrae.

The characteristics of the new bone spacer will be better clarified by the following description with reference to the drawings, which are attached as a non-restrictive example.

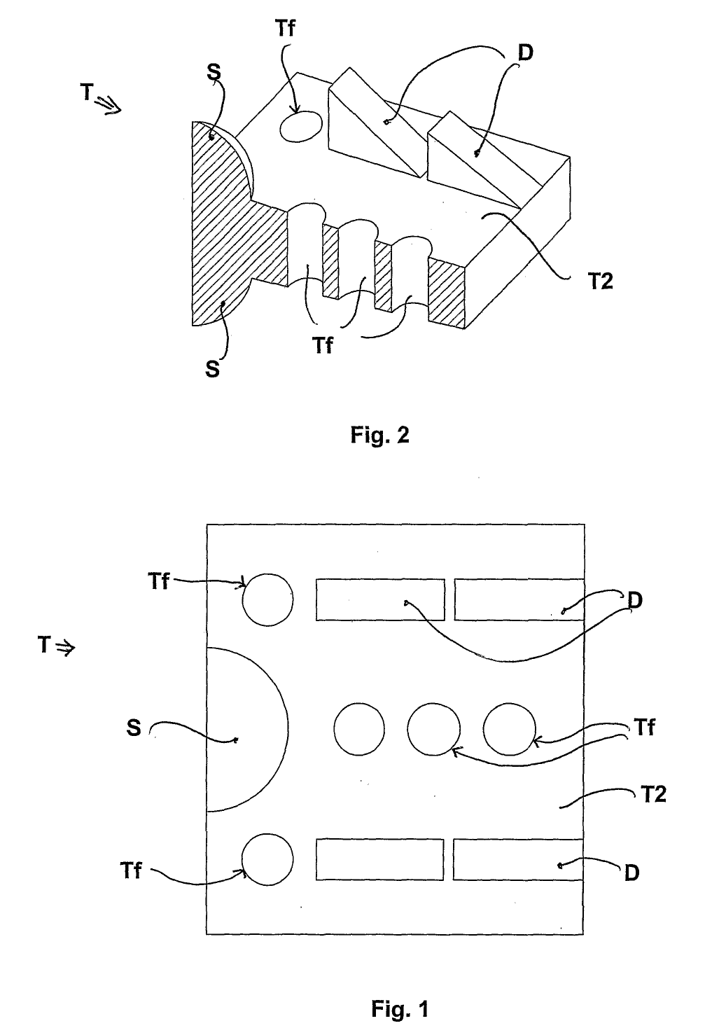

FIG. 1 shows a top view of the new spacer (T).

FIG. 2 shows a lateral cross-section of the new spacer (T).

The new bone spacer (T) consists of a device preferably in the shape of rectangular parallelepiped and preferably with a wedge-shaped profile, and with semispherically-shaped projecting parts (S) on one or both, superior and inferior, major surfaces (T2), that occupy a medial position near one edge of the spacer (T) and are suitable for being inserted and engaging inside specifically-created cavities on the surface of the vertebrae, and with further projections, or teeth (D), that prevent any displacement of said new spacer (T).

To be more specific, each major surface (T2) includes at least one semispherical projecting part (S), suitable for ensuring the easy and stable insertion of said spacer (T).

Said semispherical part (S) is designed to engage in a cavity specifically created by the operator on the vertebra before fitting said spacer (T).

Further retention devices are also provided on said major surfaces (T2) of said new spacer (T), in the form of projections, or teeth (D), to improve the immobilization of said spacer (T).

Said spacer (T) contains holes (Tf) that place the two major surfaces (T2) in communication and allow for blood flow and the formation of trabeculae.

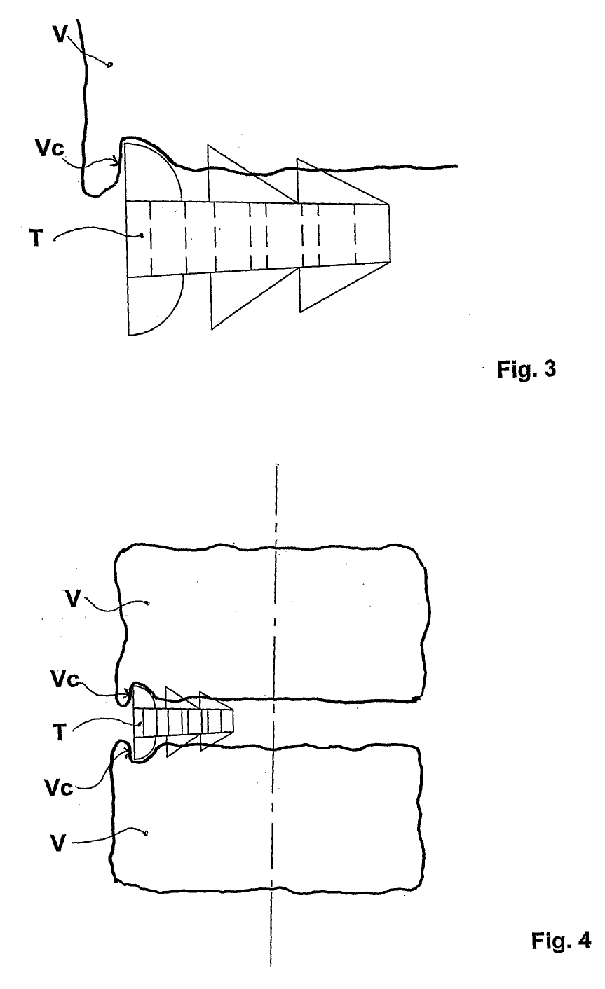

FIGS. 3 and 4 show how said spacer (T) fits between two vertebrae (V). In the case illustrated, the spacer (T) is of the symmetrical type. Each major surface (T2) is consequently complete, in corresponding positions, with a semispherical projecting part (S), holes (Tf), and further projections (D), which have a triangular shape in this case.

The semispherical projecting part (S) on each surface (T2) fits and permanently engages inside the cavities (Vc) prepared in the vertebrae (V). The projections (D) further prevent any slipping of the spacer (T) with respect to the vertebrae (V).

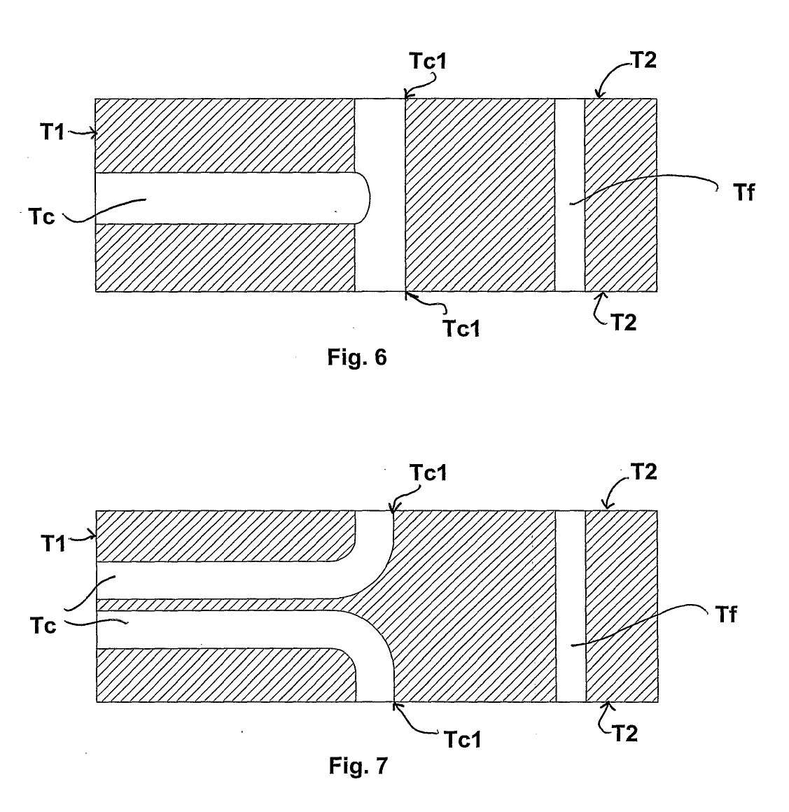

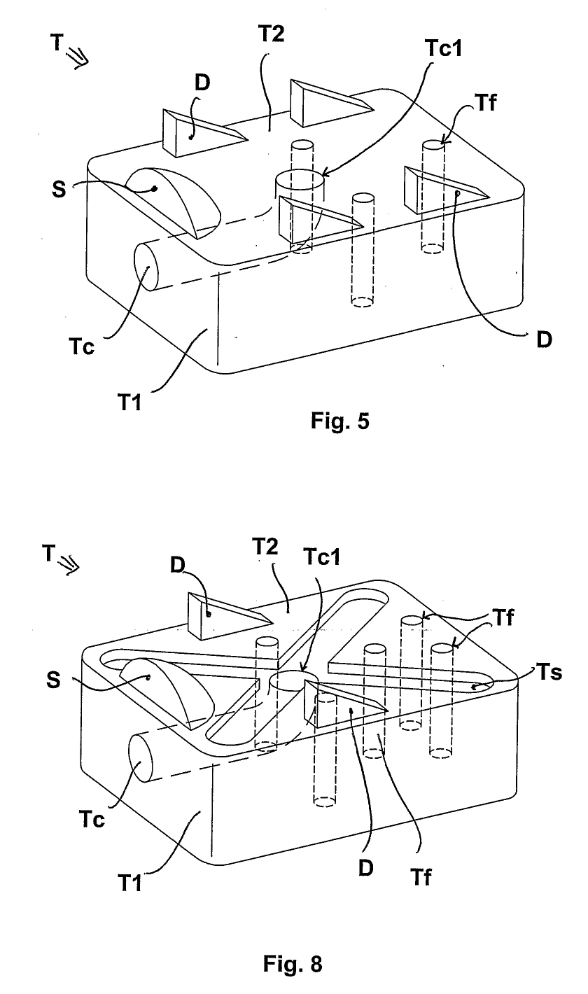

FIG. 5 shows an axonometric view of another embodiment of the new spacer (T).

Said spacer (T) also has one or more channels (Tc) placing one of the minor sides (T1) in communication with the two major sides (T2).

Basically, said channels (Tc) place the minor side (T1) of the new spacer (T), that remains accessible to the operator after the spacer has been inserted between the vertebrae, in communication with the major surfaces (T2) facing the vertebrae between which the new spacer (T) is installed.

Provision may be made for a single T-shaped channel (Tc) connecting said minor side (T1) to the two major surfaces (T2) of the new spacer (T), as illustrated in FIG. 6, or for two separate L-shaped channels (Tc), each connected to one of the two major surfaces (T2), as illustrated in FIG. 7, or for a single channel (Tc) connected to only one major surface (T2).

FIG. 8 shows another embodiment of the new spacer (T), which has radial grooves or cavities (Ts), on each surface (T2) facing the vertebrae, communicating with the outlet (Tc1) of said channels (Tc).

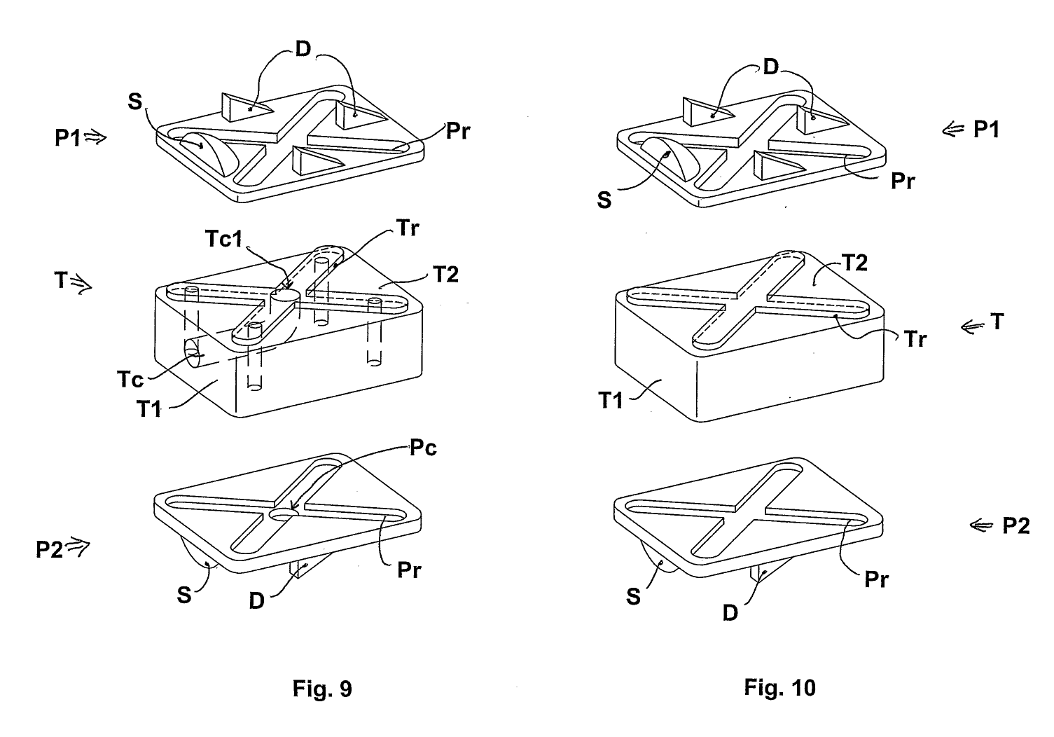

FIG. 9 shows a further embodiment of the new spacer (T) having only ribbing (Tr) on the surface (T2) facing towards the vertebrae, for coupling and centering two plates (P).

Said plates (P) have grooves (Pr), either contained within their thickness (plate P2) or penetrating through their full thickness (plate P1), for coupling with the spacer (T) and preventing it from slipping between the two surfaces (T2).

In the case of the plate (P2) with grooves (Pr) contained within its thickness, there are also through holes (Pc) aligned with the outlet (Tc1) of the channels (Tc) in the spacer (T).

There are semispherical projecting parts (S) and further projections, or teeth (D) on one or both of said plates (P1, P2).

In this example, the through holes (Tf) communicating with the two major surfaces (T2) are placed in line with the ribbing (Tr) on the spacer (T).

Provision can be made for said centering and retaining ribbing to be on the plates (P), while corresponding grooves are provided on the spacer (T).

FIG. 10 shows a further embodiment of the new spacer (T) with ribbing (Tr) for coupling and centering with the two plates (P), but without any inside channels (Tc).

The new spacer (T) is made of a suitable material having the same structure as bone trabeculae, e.g. hydroxyapatite mixed with plasma, bioceramic glass suitable for bone fusion, metal products of the type used in implants, medical elastic, plastic or silicone materials, synthetic and natural polymers, resin tissue, polyethylene, carbon and/or other materials suitable for accelerating bone fusion and orienting its vertical growth and/or as shock absorbers between the two plates (P).

The plates (P) coupled with said spacer are preferably made of titanium, steel, ceramics or other materials suitable for facilitating and favoring bone or cartilage fusion.

The new procedure for fitting the new spacer (T) and plates (P), if any, involves the surgeon milling the adjacent vertebrae before inserting said spacer (T), so as to create a cavity in said vertebrae suitable for containing said semispherical projecting part (S).

Once the new spacer (T), and any plates (P), are in position, with said semispherical parts (S) engaged in the corresponding cavities, the spacer (T) is stable, i.e. primary immobilization is guaranteed.

If the spacer (T) being fitted is of the type with internal channels (Tc), the operator injects adhesive material and/or diversified stem cells, or other types of gel or fluid material into the aforesaid channels (Tc).

Said adhesive or other material reaches and fills the gaps between the new spacer (T) and the vertebrae between which the new spacer (T) is placed, thereby ensuring the bond between the new spacer (T), the plates (P), and the vertebrae. Therefore, with reference to the above description and the enclosed drawings, the following claims are expressed.

1. Bone spacer (T) suitable for inserting between two vertebrae (V), with two surfaces (T2) suitable for coming into contact with said two vertebrae (V), characterized in that, on at least one of said surfaces, it has one or more protuberances (S), and where said protuberances are suitable for preventing the displacement of said spacer (T) from between said vertebrae.

2. Bone spacer (T), as in claim 1, characterized in that said protuberances (S) are semispherical in shape.

3. Bone spacer (T), as in claims 1, 2, characterized in that each of said protuberances (S) is suitable for engaging inside a cavity provided for said purpose on at least one of said vertebrae and thus preventing the displacement of said spacer (T) from between said vertebrae.

4. Bone spacer (T) suitable for placing in between two vertebrae, consisting of two surfaces (T2) suitable for coming into contact with said two vertebrae, characterized in that, on at least one of said sides (T2), it has one or more sets of further projections, or teeth (D).

5. Bone spacer (T), as in claim 4, characterized in that said projections or teeth (D) are triangular in shape so as to prevent the displacement of said spacer (T) from said vertebrae.

6. Bone spacer (T), characterized in that it has semispherical protuberances (S), as in claims 1, 2, 3, and projections or teeth (D), as in claims 4, 5, and said semispherical protuberances (S) and said projections or teeth (D) are both or either arranged on one or both major surfaces (T2) of said spacer (T).

7. Bone spacer (T) as in claims 1, 2, 3, 4, 5, 6, characterized in that said protuberances (S, D) are aligned with one another.

8. Bone spacer (T), as in previous claims, characterized in that it has at least one channel (Tf) for placing at least the two major surfaces (T2) in communication with one another.

9. Bone spacer (T), as in previous claims, characterized in that it is composed of a spongy material suitable for placing in communication at least the two major sides (T2) of said spacer.

10. Bone spacer (T), as in previous claims, characterized in that it includes at least one channel (Tc) suitable for placing one or both the major surfaces (T2) of said new spacer (T), that face the vertebrae, in communication with the side (T1) of said new spacer (T) that remains accessible to the operator after inserting the new spacer (T) in between said vertebrae.

11. Bone spacer (T), as in claim 10, characterized in that it contains a single T-shaped channel (Tc) communicating with the two opposite surfaces (T2) facing the vertebrae.

12. Bone spacer (T), as in claim 10, characterized in that it contains two L-shaped channels (Tc), each communicating with one major surface (T2) facing the vertebrae.

13. Bone spacer (T), as in previous claims, characterized in that it has one or more grooves or cavities (Ts) on one or both the major surfaces (T2) facing the vertebrae.

14. Bone spacer (T) as in previous claims, characterized in that said grooves or cavities (Ts) are in communication with the outlet (Tc1) of said channels (Tc).

15. Bone spacer (T) as in previous claims, characterized in that it is symmetrical.

16. Bone spacer (T) suitable for inserting between two vertebrae, with two surfaces (T2) suitable for facing said two vertebrae, characterized in that it has one or more plates (P) suitable for coupling with one or both major surfaces (T2) of the spacer (T), wherein each plate (P) is suitable for inserting between the spacer (T) and the vertebrae.

17. Bone spacer (T), as in claim 16, characterized in that at least one of said plates (P) has at least one semispherical protuberance (S) on the side opposite the spacer (T), suitable for engaging in a suitably-prepared cavity on at least one of said vertebrae, and/or at least one triangular projection or tooth (D) so as to prevent the displacement of said spacer (T) from said vertebrae.

18. Bone spacer (T), as in claims 16, 17, characterized in that it has one or more ribs (Tr) on at least one major surface (T2) facing the vertebrae to enable the coupling and centering of said plates (P), and wherein said plates (P) have grooves (Pr) suitable for coupling with said spacer (T) and preventing any slipping of said plate with respect to the surface (T2) of the spacer (T).

19. Bone spacer (T), as in claims 16, 17, characterized in that it has one or more grooves suitable for coupling and centering said plates (P) on at least one major surface (T2) facing the vertebrae, and wherein said plates (P) have ribs suitable for coupling with said grooves in the spacer (T) and thus preventing the displacement of the plate with respect to the surface (T2) of the spacer (T).

20. Bone spacer (T), as in claims 16, 17, 18, 19, characterized in that it has at least one channel (Tc) suitable for placing one or both the major surfaces (T2) of said new spacer (T), that face the vertebrae, in communication with the side (T1) of said new spacer (T) that remains accessible to the operator after inserting the new spacer (T) between said vertebrae.

21. Bone spacer (T), as in claim 20, characterized in that it contains a single T-shaped channel (Tc) communicating with the two opposite surfaces (T2) that face the vertebrae.

22. Bone spacer (T), as in claim 20, characterized in that it contains two L-shaped channels (Tc), each communicating with one major surface (T2) facing the vertebrae.

23. Bone spacer (T), as in claims 16, 17, 18, 19, characterized in that it has one or more grooves or cavities (Ts) on one or both the major surfaces (T2) facing the vertebrae.

24. Bone spacer (T) as in claims 20, 21, 22, 23, characterized in that said grooves or cavities (Ts) are in communication with the outlet (Tc1) of said channels (Tc).

25. Bone spacer (T), as in claims 20, 21, 22, 23, characterized in that the grooves (Pr) in each plate (P) are shallower than the full thickness of said plate (P), and wherein said plates (P) also have through holes (Pc) aligned with the outlet (Tc1) of the channels (Tc) in the spacer (T).

26. Bone spacer (T), as in claims 20, 21, 22, 23, characterized in that the grooves (Pr) in each plate (P) are as deep as the full thickness of said plate (P).

27. Bone spacer (T) as in previous claims, characterized in that it is made of one of the following materials, or a combination thereof: hydroxyapatite mixed with plasma, bioceramic glass suitable for bone fusion, metal products of the type used in implants, medical elastic, plastic or silicone materials, synthetic and natural polymers, resin tissue, polyethylene, suitable for injecting cells capable of colonizing and converting the support material.

28. Bone spacer (T) as in previous claims, characterized in that said plates (P) are made of one of the following materials, or a combination thereof: titanium, steel, ceramics or other materials suitable for facilitating and favoring bone or cartilage fusion.

29. Bone spacer (T) as in claim 28, characterized in that it is coated with carbon.
